(19) United States
(12) Patent Application Publication
(10) Pub. No.: US 2013/0323776 A1
(43) Pub. Date: Dec. 5, 2013

(54) CARRIER PEPTIDE FRAGMENT AND USE THEREOF

(71) Applicant: Toagosei Co. Ltd., Tokyo (JP)

(72) Inventors: Tetsuhiko YOSHIDA, Tsukuba-shi (JP); Nahoko KOBAYASHI, Tsukuba-shi (JP); Mikio NIWA, Tsukuba-shi (JP); Kenichi TANAKA, Tsukuba-shi (JP)

(21) Appl. No.: 13/936,782

(22) Filed: Jul. 8, 2013

Related U.S. Application Data
(62) Division of application No. 13/386,585, filed on Jan. 23, 2012.

(30) Foreign Application Priority Data
Jul. 29, 2009 (JP) 2009-177102

(51) Int. Cl. G01N 33/50

(52) U.S. Cl. CPC G01N 33/5005
USPC 435/29; 435/375; 435/366; 435/347; 435/455

(57) ABSTRACT

A method for transferring a foreign substance includes: preparing a construct for transferring a foreign substance that contains a carrier peptide fragment including any amino acid sequence selected from SEQ ID NOS: 1-6, or an amino acid sequence formed by the substitution, deletion, and/or addition (insertion) of 1, 2, or 3 amino acid residues in the amino acid sequence of the selected sequence identification number, and a foreign substance of interest that is bonded to the N-terminus and/or C-terminus of the carrier peptide fragment; supplying the construct for transferring a foreign substance to a test sample that contains a target eukaryotic cell; and incubating the test sample that has been supplied with the construct for transferring a foreign substance to thereby transfer the construct into the eukaryotic cell in the test sample.

This is a divisional of application Ser. No. 13/386,585 filed Jan. 23, 2012, which is a National Stage Application of PCT/JP2010/062692 filed Jul. 28, 2010, and claims the benefit of Japanese Application No. 2009-177102 filed Jul. 29, 2009. The entire disclosures of the prior applications are hereby incorporated by reference herein in their entirety.

### TECHNICAL FIELD

The present invention relates to a method for transferring (carrying) a foreign substance from outside a eukaryotic cell into the cell, and a carrier peptide fragment used in the method.

The present application claims priority on the basis of Japanese Patent Application No. 2009-177102 filed on 29 Jul. 2009, and the entire content of the domestic application is incorporated into the description of the present application by reference.

### BACKGROUND ART

Polypeptides and other foreign substances, particularly biologically active substances, are transferred into the cells of humans and other mammals, etc., (eukaryotic cells) to change the characteristics or to improve and enhance the function of the cells (as well as the tissues and organs comprising the cells).

For example, Patent Document 1 discloses a transcellular carrier peptide for transferring polypeptide, DNA or another foreign substance into a cell. This patent indicates that a polypeptide, DNA, or other biologically active substance can be transferred into a cell with high efficiency by using a carrier peptide conjugate comprising a transcellular carrier peptide linked to a xenogenic polypeptide, DNA, and the like.

Still, a method is needed for changing the characteristics and improving (or enhancing) the function of the cells by easily transferring a full-length polypeptide with a relatively large molecular weight as the foreign substance (biologically active substance) to be transferred into a target cell without the use of special equipment.

Alternatively, in place of transferring a polypeptide or a full-length protein, a method is needed wherein the focus is placed on the specific function of the polypeptide, and a partial amino acid sequence that is the minimum unit capable of expressing that function, i.e., an amino acid sequence (foreign substance) constituting a peptide motif, is transferred efficiently into the cell.

For example, Patent Document 2 discloses part of a peptide chain (amino acid sequence) found in the SOCS protein and other proteins of the same family (hereinafter, “SOCS proteins”) that is a motif wherein the amino acid sequence constituting all or part of the specific region called the “BC box,” which is believed to bind to the elongin BC complex, has a high level of neuronal differentiation inducing activity on somatic stem cells. Patent Document 2 also discloses that transferring the motif into mammalian somatic stem cells can induce them to differentiate into nerve cells.
Patent Document 1: Japanese Patent Publication No. 3854995
Patent Document 2: WO 2007/010989
Patent Document 3: Japanese Patent Application Laid-open No. 2005-330206
Non-Patent Document 1: EMBO Reports, Vol. 10, No. 3, 2009, pages 231 to 238
Non-Patent Document 2: PNAS, Vol. 95, 1998, pages 114 to 119
Non-Patent Document 3: Genes & Development, Vol. 12, 1998, pages 3872 to 3881
Non-Patent Document 4: Genes & Development, Vol. 18, 2004, pages 2867 to 2872
Non-Patent Document 5: Genes & Development, Vol. 18, 2004, pages 3055 to 3065

### DISCLOSURE OF THE INVENTION

However, when a peptide-based foreign substance such as a polypeptide, protein, or peptide motif that has a certain function, or a foreign substance other than a peptide (such as a nucleic acid, etc.) is transferred into a cell, the site (or organelle) to which the foreign substance of interest can transferred has been a major problem. The reason is that the extent of the effect thereof on the recipient cell is believed to differ considerably depending on the site to which it is translocated. For example, if a motif having a function related to cellular differentiation and transformation such as the abovementioned neurodifferentiation is transferred, there are cases wherein it is desirable for the motif to be transferred (transported) not only into the cytoplasm, but additionally into the nucleus.

Hence, the prevent invention was created in response to this need, and an object of the present invention is to provide a carrier peptide fragment that is used to transfer a foreign substance from outside a cell (typically a eukaryotic cell, and particularly an animal cell from a mammal, etc., that does not have a cell wall) into the cell wherein the peptide fragment itself is a sequence motif that inherently directs translocation into the nucleus. Another object of the present invention is to provide a method that uses this carrier peptide fragment to pass a variety of foreign substances through the cell membrane from outside and transfer the same into a target cell (and typically, also into the nucleus). Moreover, the present invention provides a construct for transferring a foreign substance that has been configured to comprise the carrier peptide fragment disclosed herein and a foreign substance. Furthermore, the present invention provides a cell, organ, or other biological tissue obtained by transferring the construct comprising the carrier peptide fragment disclosed herein and a foreign substance into the cytoplasm (typically, also into the nucleus) thereof.

The inventors conducted various investigations of peptides with previously identified amino acid sequences (or amino acid sequences constituting parts of peptides (i.e., motifs with identified functions)) as peptides that have some kind of intracellular function, and they discovered that some nucleolar localization signals (hereinafter abbreviated as “NoLS”), which are known to be signal sequences that localize a protein into the nucleolus within the nucleus (Non-Patent Document 1), can be used as amino acid sequences (sequence motifs) that provide a heretofore unexpected function, thus completing the present invention.

More specifically, the inventors discovered that some of the NoLS will function as a carrier peptide that can independently pass through the cell membrane from outside the cell and transfer a foreign substance into the cytoplasm, thus completing the present invention.

One method provided by the present invention is a method for transferring (carrying) a foreign substance of interest from outside (i.e., outside the cell membrane) of eukaryotic cells (in particular, various animal cells typified by human and other mammalian cells that do not have a cell wall) at least into the cytoplasm thereof.

More specifically, the method for transferring a foreign substance disclosed herein comprises the steps of:

preparing a construct for transferring a foreign substance that contains a carrier peptide fragment comprising any amino acid sequence known as a nucleolar localization signal (NoLS) and selected from. SEQ ID NOS: 1, 2, 3, 4, 5, and 6, or an amino acid sequence formed by the substitution, deletion, and/or addition (insertion) of 1, 2, or 3 amino acid residues in the amino acid sequence of the selected sequence identification number, and a foreign substance of interest that is bonded to the N-terminus and/or C-terminus of the carrier peptide fragment;

supplying the construct for transferring a foreign substance to a test sample that contains a target eukaryotic cell; and

incubating the test sample that has been supplied with the construct for transferring a foreign substance (i.e., maintaining the test sample under conditions enabling survival of the cell of interest for a predetermined time period) to thereby transfer the construct into the eukaryotic cell in the test sample.

The term “foreign substance” used herein refers to an inorganic or organic compound that is capable of bonding either directly or indirectly via a suitable linker to the N-terminus or C-terminus of the abovementioned carrier peptide fragment, and that has a molecular size and chemical properties that make the transfer thereof into a eukaryotic cell possible.

The transfer method of the present invention with the abovementioned configuration enables a foreign substance of interest to pass through the cell membrane from outside a eukaryotic cell (outside the cell membrane) and be carried into the cytoplasm (more preferably, pass through the nuclear membrane and into the nucleus) with high efficiency by preparing a construct for transferring a foreign substance by bonding a foreign substance of interest (typically, an organic chemical such as a peptide, nucleic acid, dye, drug, etc.) either directly or indirectly via a suitable linker to the N-terminus and/or C-terminus of a carrier peptide (fragment) with an amino acid sequence defined by any of the above-mentioned sequence identification numbers and supplying that construct to a test sample containing a eukaryotic cell of interest (typically a culture containing the cell) (in other words, by adding the construct to a living eukaryotic cell).

In one preferred mode of the method for transferring a foreign substance disclosed herein, the abovementioned foreign substance is characterized in that it is any organic compound selected from a group consisting of peptides, nucleic acids, dyes, and drugs.

The term “peptide” used herein refers to an organic compound with a structure wherein two or more amino acids are joined by peptide bonds, and it includes polypeptides (typically, at least 10 but fewer than 300 amino acid residues) and proteins (typically, macromolecular compounds comprising a larger number (300 or more) of amino acid residues than the abovementioned polypeptides).

Moreover, the term “nucleic acid” used herein refers to a nucleotide polymer and includes DNA and RNA.

A construct prepared so that it contains this type of organic compound enables the transfer thereof into the target cell with good efficiency.

Moreover, in another preferred mode of the method for transferring a foreign substance disclosed herein, the abovementioned foreign substance is a peptide, and the abovementioned construct for transferring a foreign substance is a synthetic peptide containing a fragment from a peptide serving as the foreign substance and the abovementioned carrier peptide fragment.

The method of this mode enables a peptide of interest (i.e., the amino acid sequence constituting the peptide) to be transferred into the target cell as a peptide motif in the form of the abovementioned synthetic peptide.

In another preferred mode of the method for transferring a foreign substance disclosed herein, the eukaryotic cell that is the target to which the abovementioned construct for transferring a foreign substance is to be transferred is characterized in that it is a stem cell (including an induced pluripotent stem cell here and hereinafter) derived from a human or other nonhuman mammal.

The present invention enables the transfer of a foreign substance of interest having a designated function into a human or other mammalian stem cell (for example, a somatic stem cell or induced pluripotent stem cell). As a result, the stem cell can be transformed in response to the transferred foreign substance (peptide, etc.), and for example, can differentiate into a specific cell type (nerve cell, bone cell, muscle cell, skin cell, etc.).

Moreover, to realize the abovementioned object, the present invention provides a construct artificially prepared in order to transfer a foreign substance of interest from outside a eukaryotic cell (in particular, various animal cells typified by human and nonhuman mammalian cells that do not have a cell wall) into the cytoplasm (preferably, also into the nucleus) thereof.

In other words, the construct for transferring a foreign substance disclosed herein contains a carrier peptide fragment comprising any amino acid sequence known as a nucleolar localization signal (NoLS) and selected from SEQ ID NOS: 1, 2, 3, 4, 5, and 6, or an amino acid sequence formed by the substitution, deletion, and/or addition (insertion) of 1, 2, or 3 amino acid residues in the amino acid sequence of the selected sequence identification number, and a foreign substance of interest that is bonded to the N-terminus and/or C-terminus of the carrier peptide fragment.

A foreign substance of interest can be transferred effectively to a target cell by performing the transfer method for a foreign substance of the present invention utilizing this construct. In addition, cells wherein the foreign substance has been transferred into the cytoplasm (or preferably, into the nucleus), as well as organs and other body tissues comprising cells containing the foreign substance can be obtained thereby.

Preferably, as noted above, the abovementioned oned foreign substance is any organic compound selected from a group consisting of peptides, nucleic acids, dyes, and drugs.

Moreover, most preferably the abovementioned foreign substance is a peptide, and the abovementioned construct for transferring a foreign substance is a synthetic peptide containing a fragment from a peptide serving as the foreign substance and the abovementioned carrier peptide fragment.

### BRIEF DESCRIPTION OF THE DRAWINGS

FIG. 1 is a micrograph taken while using a confocal laser scanning microscope to observe a specimen (cells) after human neonate foreskin fibroblasts had been supplied with the construct for transferring a foreign substance (Sample No. 14) as in one example, cultured for 1 hour, and fixed in methanol; the scale in the photo representing 100 μm.

FIG. 2 is a micrograph taken while using a confocal laser scanning microscope to observe a specimen (cells) after human neonate foreskin fibroblasts had been supplied with the construct for transferring a foreign substance (Sample No. 4) as in one example, cultured for 1 hour, and fixed in methanol; the scale in the photo representing 100 μm.

FIG. 3 is a micrograph taken while using a confocal laser scanning microscope to observe a specimen (cells) after human neonate foreskin fibroblasts had been supplied with the construct for transferring a foreign substance (Sample No. 5) as in one example, cultured for 1 hour, and fixed in methanol; the scale in the photo representing 100 μm.

FIG. 4 is a micrograph taken while using a confocal laser scanning microscope to observe a specimen (cells) after human neonate foreskin fibroblasts supplied with the construct for transferring a foreign substance (Sample No. 6) related in one example had been cultured for 1 hour and fixed in methanol; the scale in the photo represents 100 μm.

FIG. 5 is a micrograph taken while using a confocal laser scanning microscope to observe a specimen (cells) after human neonate foreskin fibroblasts supplied with the construct for transferring a foreign substance (Sample No. 14) related in one example had been cultured for 4 hours and fixed in methanol; the scale in the photo represents 100 μm.

FIG. 6 is a micrograph taken while using a confocal laser scanning microscope to observe a specimen (cells) after human neonate foreskin fibroblasts supplied with the construct for transferring a foreign substance (Sample No. 2) related in one example had been cultured for 4 hours and fixed in methanol; the scale in the photo represents 100 μm.

FIG. 7 is a micrograph taken while using a confocal laser scanning microscope to observe a specimen (cells) after human neonate foreskin fibroblasts supplied with the construct for transferring a foreign substance (Sample No. 3) related in one example had been cultured for 4 hours and fixed in methanol; the scale in the photo represents 100 μm.

FIG. 8 is a micrograph taken while using a confocal laser scanning microscope to observe a specimen (cells) after human neonate foreskin fibroblasts supplied with the construct for transferring a foreign substance (Sample No. 4) related in one example had been cultured for 4 hours and fixed in methanol; the scale in the photo represents 100 μm.

FIG. 9 is a micrograph taken while using a confocal laser scanning microscope to observe a specimen (cells) after human iPS cells supplied with the construct for transferring a foreign substance (Sample No. 14) related in one example had been cultured for 1 hour and fixed in methanol; the scale in the photo represents 100 μm.

FIG. 10 is a micrograph taken while using a confocal laser scanning microscope to observe a specimen (cells) after human iPS cells supplied with the construct for transferring a foreign substance (Sample No. 4) related in one example had been cultured for 1 hour and fixed in methanol; the scale in the photo represents 100 μm.

FIG. 11 is a micrograph taken while using a confocal laser scanning microscope to observe a specimen (cells) after human iPS cells supplied with the construct for transferring a foreign substance (Sample No. 6) related in one example had been cultured for 1 hour and fixed in methanol; the scale in the photo represents 100 μm.

FIG. 12 is a micrograph taken while using a confocal laser scanning microscope to observe a specimen (cells) after human iPS cells supplied with the construct for transferring a foreign substance (Sample No. 7) related in one comparative example had been cultured for 1 hour and fixed in methanol; the scale in the photo represents 100 μm.

FIG. 13 is a micrograph taken while using a confocal laser scanning microscope to observe a specimen (cells) after human iPS cells supplied with the construct for transferring a foreign substance (Sample No. 8) related in one comparative example had been cultured for 1 hour and fixed in methanol; the scale in the photo represents 100 μm.

### BEST MODE FOR CARRYING OUT THE INVENTION

Preferred embodiments of the invention of the present invention are described below. It should also be noted that matters necessary for carrying out the invention beyond those specifically stated in the present description (for example, general matters related to peptide synthesis and cell culture) are understood to be matters of design based on prior art in fields such as medicine, pharmacology, organic chemistry, biochemistry, genetic engineering, protein synthesis, molecular biology, hygiene, and the like.

Moreover, the present invention can be carried out on the basis of the details disclosed herein and common technical knowledge in the fields. It should also be noted that in each instance the amino acids are expressed in the following explanation by single letter codes (by 3-letter codes in the sequence listings) based on the nomenclature for amino acids in the IUPAC-IUB guidelines.

The term “carrier peptide fragment” of the present invention disclosed herein is a sequence defined by any of the amino acid sequences selected from abovementioned SEQ ID NOS: 1 to 6 (or a modified sequence thereof), and is an amino acid sequence that exhibits cell membrane permeability (more preferably nuclear localization capability (i.e., nuclear membrane permeability)) in eukaryotic cells. Alternatively, the term “carrier peptide fragment” of the present invention is a sequence defined by any of the amino acid sequences selected from abovementioned SEQ ID NOS: 9 to 13 (or a modified sequence thereof), and is an amino acid sequence that exhibits cell membrane permeability (more preferably nuclear localization capability (i.e., nuclear membrane permeability)) in eukaryotic cells.

In this case, the amino acid sequence of SEQ ID NO: 1 corresponds to an NoLS comprising a total of 14 amino acid residues derived from FGF2 (basic fibroblast growth factor).

The amino acid sequence of SEQ ID NO: 2 corresponds to an NoLS comprising a total of 19 amino acid residues derived from one of the nucleolar proteins (ApLLP).

The amino acid sequence of SEQ ID NO: 3 corresponds to an NoLS comprising a total of 16 amino acid residues derived from a protein (γ(1)34.5) of HSV-1 (herpes simplex virus type 1).

The amino acid sequence of SEQ ID NO: 4 corresponds to an NoLS comprising a total of 19 amino acid residues derived from the p40 protein of HIC (human I-mfa domain-containing protein).

The amino acid sequence of SEQ ID NO: 5 corresponds to an NoLS comprising a total of 16 amino acid residues derived from the MEQ protein of MDV (Marek disease virus).

The amino acid sequence of SEQ ID NO: 6 corresponds to an NoLS comprising a total of 17 amino acid residues derived from apoptosis-inhibiting protein Survivin-deltaEx3.

The amino acid sequence of SEQ ID NO: 7 corresponds to an NoLS comprising a total of 7 amino acid residues derived from the vascular growth factor angiogenin.

The amino acid sequence of SEQ ID NO: 8 corresponds to an NoLS comprising a total of 8 amino acid residues derived from MDM2, which forms a complex with the p53 tumor suppressor protein, a nuclear phosphoprotein.

The amino acid sequence of SEQ ID NO: 9 corresponds to an NoLS comprising a total of 9 amino acid residues derived from GGNNVa, a betanodavirus protein.

The amino acid sequence of SEQ ID NO: 10 corresponds to an NoLS comprising a total of 7 amino acid residues derived from NF-izB inducing kinase (NIK).

The amino acid sequence of SEQ ID NO: 11 corresponds to an NoLS comprising a total of 15 amino acid residues derived from Nuclear VCP-like protein.

The amino acid sequence of SEQ ID NO: 12 corresponds to an NoLS comprising a total of 18 amino acid residues derived from the nucleolar protein p120.

The amino acid sequence of SEQ ID NO: 13 corresponds to an NoLS comprising a total of 14 amino acid residues derived from the ORF57 protein of HVS (herpes virus saimiri).

The “carrier peptide fragment” disclosed herein is typically a sequence identical to the amino acid sequences represented by SEQ ID NO: 1 to 6 (or SEQ ID NO: 9, 10, 11, 12 or 13), but in addition thereto, it encompasses an amino acid sequence formed by the substitution, deletion and/or addition (insertion) of 1 or several (typically 2 or 3) amino acid residues therein without the loss of cell membrane permeability. In other words, such a slightly modified sequence can be easily used by a person skilled in the art on the basis of the information disclosed herein, and therefore is encompassed by the term “carrier peptide fragment” as a technical concept disclosed herein. Typical examples include a sequence produced by so-called conservative amino acid replacement wherein 1 or several (typically 2 or 3) amino acid residues in the amino acid sequence of SEQ ID NO: 1 are conservatively replaced (for example, a sequence wherein a basic amino acid residue is replaced by a different basic amino acid residue), or a sequence wherein 1 or several (typically 2 or 3) amino acid residues are added (inserted) to or deleted from the designated amino acid sequence.

The construct for transferring a foreign substance disclosed herein is a construct that can be designed and configured by bonding (linking), either directly or indirectly via a suitable linker, a desired foreign substance to the N-terminus and/or C-terminus of the abovementioned carrier peptide fragment. For example, if the foreign substance is a peptide, the peptide chain can be designed to contain the amino acid sequence constituting the peptide and the amino acid sequence constituting the carrier peptide fragment, and then the intended construct for transferring a foreign substance of interest can be prepared by synthesizing the peptide chain. Moreover, the construct for transferring a foreign substance can be configured by directly or indirectly bonding a nucleic acid such as various types of DNA or RNA, or an organic compound that acts as a dye (for example, a fluorescent compound such as FITC) or that acts as a drug (for example a nucleic acid-based anticancer drug such as 5-fluorouracil (SFU) or an antiviral drug such as azidothymidine (AZT)) to the N-terminus and/or C-terminus of the above carrier peptide fragment by various prior art and publicly known chemical methods.

It should also be noted that when the foreign substance is a peptide, the peptide (amino acid sequence) to be used is not particularly limited herein. A polypeptide with a relatively large number of amino acid residues, for example about 100 to 1000 amino acid residues, can be used as the foreign substance.

Typically, a suitable number for the total number of amino acid residues constituting the synthetic peptide prepared as the construct for transferring a foreign substance is 1000 or fewer, preferably 600 or fewer, and even more preferably 500 or fewer, and most preferably 300 or fewer (and further, 100 or fewer, e.g., 10 to 50). Such a relatively short peptide is easy to synthesize and easy to use.

Preferably, the foreign substance to be used is a mature form or precursor (including pro-forms and prepro-forms) of a peptide (including polypeptides and proteins) involved in a function such as the development, differentiation, growth, malignant transformation, homeostasis, and regulation of metabolism in various cells and tissues (organs). Moreover, the present invention can be carried out to transfer a peptide with a heretofore unknown function into a cell to elucidate the function of the peptide with the cell (within a biological tissue).

For example, when the eukaryotic cell that is the target of transfer is a human or other mammalian stem cell (including somatic stem cells, embryonic stem cells, and induced pluripotent stem cells (hereinafter, iPS cells)), preferably the mature form or precursor of a peptide with various types of biological activity involving the induction of differentiation of the stem cell will be used. Moreover, when the eukaryotic cell that is the target of transfer is a cancer cell (tumor cell), preferably various peptides involved in the induction of apoptosis of the cancer cell (tumor cell) will be used.

Alternatively, in the past iPS cells have been prepared by transducing a plurality of genes (for example, Oct3/4, Sox2, Klf4, c-Myc, Nanog, Lin28) into a designated cell (for example, a human or other mammalian skin cell or other somatic cell), and at least one gene product (peptide) from among these genes can be transferred by the transfer method of the present invention in place of the technique. Thus, it will be possible to prepare iPS cells by transferring the products of the abovementioned genes (i.e., peptides) into the cells (preferably the nucleus) in place of the direct transduction of the genes.

Therefore, an example of one preferred embodiment of the present invention is a method for preparing iPS cells wherein the construct for transferring a foreign substance of the present invention is prepared using as the foreign substance a peptide (for example Sox2 protein), encoded by at least one of a plurality of genes (for example Sox2) involved in the preparation of iPS cells, and the construct is then transferred into a designated eucaryotic cell (such as a human dermal fibroblast, etc.).

Moreover, a variety of previously known sequence motifs (peptide motifs) can be used therefor. For example, when the eukaryotic cell that is the target of transfer is a human or other mammalian stem cell (including somatic stem cells, embryonic stem cells, and iPS cells), preferably a variety of peptide motifs involved in inducing differentiation of the stem cell will be used. Moreover, when the eukaryotic cell that is the target of transfer is a cancer cell (tumor cell), preferably various peptide motifs involved in inducing apoptosis of the cancer cell (tumor cell) will be used.

Prime examples of peptide motifs that can be used most preferably in the examples of the present application include the various amino acid sequences (motifs) disclosed in Patent Document 2 above that exhibit neurodifferentiation properties.

In other words, Patent Document 2 discloses partial amino acid sequences constituting the various SOCS (suppressor of cytokine signaling) proteins and other proteins of the same family (hereinafter, “SOCS proteins”) that all have a SOCS-box, which is a region (amino acid sequence) that can bind to the elongin BC complex (specifically, a part of elongin C), which is known to form a complex with elongin A and act as a transcription regulating factor. Patent Document 2 also indicates that this amino acid sequence, which is contained in a specific region called the “BC-box” that is believed to bind with the elongin BC complex, has a high level of neurodifferentiation inducing activity in somatic stem cells.

SEQ ID NOS: 14 to 31 are typical examples of amino acid sequences that are contained in the BC-box of various proteins identified as SOCS proteins (see Non-Patent Documents 2 to 5).

More specifically, these represent amino acid sequences comprising 15 contiguous amino acid residues from the N-terminus of the BC-boxes contained in mSOCS-1 (SEQ ID NO: 14), mSOCS-2 (SEQ ID NO: 15), mSOCS-3 (SEQ ID NO: 16), mSOCS-4 (SEQ ID NO: 17), mSOCS-5 (SEQ ID NO: 18), hSOCS-6 (SEQ ID NO: 19), hSOCS-7 (SEQ ID NO: 20), hRAR-1 (SEQ ID NO: 21), hRAR-like (SEQ ID NO: 22), mWSB-1 (SEQ ID NO: 23), mWSB-2 (SEQ ID NO: 24), mASB-1 (SEQ ID NO: 25), mASB-2 (SEQ ID NO: 26), hASB-3 (SEQ ID NO: 27), LRR-1 (SEQ ID NO: 28), hASB-7 (SEQ ID NO: 29), mASB-10 (SEQ ID NO: 30) and hASB-14 (SEQ ID NO: 31) (see Non-Patent Documents 2 to 5).

Moreover, although a specifically detailed explanation is omitted herein, SEQ ID NOS: 32 to 92 represent amino acid sequences contained in the BC-boxes of various SOCS proteins identified in viruses (HIV, AdV, SIV, etc.) and in mammals, and the peptides comprising the sequences. For example, SEQ ID NOS: 87 and 91 are amino acid sequences contained in the BC-box of a SOCS protein (MUF1) identified from humans. Moreover, SEQ ID NO: 92 is the amino acid sequence contained in the BC-box of a SOCS protein mCIS (cytokine-inducible SH₂-containing protein) identified from mice.

These are merely examples, and there is no intention herein to limit the amino acid sequences (motifs) constituting the BC-box to these sequences. When the present application was filed amino acid sequences constituting various BC-boxes had been disclosed in a number of published documents and there is no need for further listing of examples herein. These amino acid sequences can be easily found through conventional search methods.

In one preferred embodiment of the present invention, the synthetic peptide to be transferred to a target eukaryotic cell (for example, a somatic stem cell from a human or nonhuman mammal) can be configured using any of the above amino acid sequences originating in the BC-box (typically any amino acid sequence from SEQ ID NOS: 14 to 92) as a peptide motif (sequence motif) that is involved in inducing neurodifferentiation. Therefore, in accordance with the abovementioned explanation, the present invention provides a method for inducing the differentiation of at least one type of eukaryotic cell into a nerve cell. In other words, this method includes the steps of first synthesizing a peptide chain featuring an amino acid sequence originating in any BC-box (typically a sequence comprising at least 10 contiguous amino acid residues (for example at least 10 residues starting from the N-terminus) selected from any amino acid sequence represented by SEQ ID NOS: 14 to 92) as a peptide motif involved in inducing neurodifferentiation bonded to the N-terminal end or C-terminal end of the abovementioned carrier peptide fragment according to the present invention, and then supplying the synthetic peptide (i.e., an artificial peptide that is the construct for transferring a foreign substance) to a test sample containing a target eukaryotic cell or tissue comprising the cell (typically a culture product containing the cell). Typically, this also includes incubation of the test sample to which the synthetic peptide is supplied.

Moreover, it is clear from the abovementioned explanation that the present invention provides an artificial peptide used in a method for inducing such differentiation to a nerve cell and a method for preparing the same.

In other words, the artificial peptide of this configuration (neurodifferentiation-inducing peptide) can be synthesized so that it provides an amino acid sequence originating in any BC-box (hereinafter called a “BC-box-related sequence” and typically a sequence comprising at least 10 contiguous amino acid residues (for example at least 10 residues starting from the N-terminus) selected from any amino acid sequence represented by SEQ ID NOS: 14 to 92) as a peptide motif involved in inducing neurodifferentiation bonded onto the N-terminal end or C-terminal end of the abovementioned carrier peptide fragment.

Alternatively, the amino acid sequence of 15 contiguous amino acid residues represented by SEQ ID NO: 93 can be used for the same purpose as the abovementioned BC-box-related sequence. In other words, as disclosed in Patent Document 3, the amino acid sequence represented by SEQ ID NO: 93 is a partial amino acid sequence comprising 15 contiguous amino acid residues from residues 157 to 171 of the amino acid sequence of the Von Hippel-Lindau (VHL) protein, which is known to exhibit neurodifferentiation-inducing capability (i.e., SEQ ID NO: 81 is a VHL-related peptide motif).

Furthermore, just as in the case of the carrier peptide fragment of the present invention disclosed above, it is surely possible to use a modified amino acid sequence or peptide motif (foreign substance) involved in inducing neurodifferentiation that is formed by the replacement, deletion, and/or addition (insertion) of 1 or several (for example, 5 or less, and typically 2 or 3) amino acid residues therein provided its function as a peptide motif related to inducing neurodifferentiation is retained.

The construct for transferring a foreign substance with the above-mentioned configuration has a high level of neurodifferentiation-inducing activity toward at least one type of cell (typically a stem cell) as a neurodifferentiation-inducing peptide. Hence, it can most suitably be used as an active ingredient in a neurodifferentiation-inducing agent. It should be noted that the neurodifferentiation-inducing peptide contained in the neurodifferentiation-inducing agent can also take the form of a salt provided the neurodifferentiation-inducing activity thereof is not lost. For example, an acid addition salt of the peptide that is obtained by carrying out an addition reaction with a conventionally used inorganic or organic acid by conventional means can be used therefor. Alternatively, a different salt (for example, a metal salt) can be used provided it has neurodifferentiation-inducing activity.

The neurodifferentiation-inducing agent can contain a neurodifferentiation-inducing peptide of the abovementioned constitution as the active ingredient, as well as various medically (pharmaceutically) permissible carriers in accordance with the form of use. A carrier generally used in peptide medicines is preferably used as a diluent, excipient, and the like. The carrier will differ appropriately in accordance with the usage and form of the neurodifferentiation-inducing agent, but typical examples include water, a physiological buffer solution, and various organic solvents. The carrier can be an aqueous solution of alcohol (ethanol, etc.) at a suitable concentration, glycerol, or a non-drying oil such as olive oil. Alternatively, the carrier can be a liposome. Examples of a secondary ingredients that can be contained in the neurodifferentiation-inducing agent include various fillers, expanders, binders, moisturizers, surfactants, pigments, fragrances, etc.

The form of the neurodifferentiation-inducing agent is not particularly limited herein. Examples of typical forms include liquids, suspensions, emulsions, aerosols, foams, granules, powders, tablets, capsules, and ointments. Moreover, the agent can also be made into a lyophilized product or granulated product to be dissolved in physiological saline or a suitable buffer (e.g., PBS), etc., immediately before use and prepared as a liquid for injection, etc.

It should also be noted that prior art, publicly known methods can be used for the processes themselves whereby the neurodifferentiation-inducing peptide (main ingredient) and various carriers (secondary ingredients) are made into a material and then prepared as the medicines (compositions) in various forms, and a detailed explanation of the production process for drug product formulation itself is omitted herein because it is not a characterizing feature of the present invention. For example, Comprehensive Medicinal Chemistry, edited by Corwin Hansch, Pergamon Press, 1990, can be noted as a source of detailed information concerning formulations.

The dosage and administration of the neurodifferentiation-inducing agent provided by the present invention can be suited to the form and purpose thereof.

For example, exactly the desired amount of the neurodifferentiation-inducing peptide synthesized to contain a BC-box-related sequence or VHL peptide motif and the carrier peptide fragment disclosed herein (in other words, the neurodifferentiation-inducing agent comprising the synthetic peptide) can be administered as a liquid medicine to a patient (i.e., to the body) by intravenous, intramuscular, subdermal, intradermal, or intraperitoneal injection. Alternatively, it can be administered orally in solid form such as a tablet, etc. Thus, typically neurons can be generated (produced) in vivo from somatic stem cells present at or near the diseased area. As a result, nerve regeneration can serve as a powerful therapeutic method that can effectively treat a variety of neurological disorders. For example, treatment of neurological disorders such as Parkinson's disease, cerebral infarction, Alzheimer's disease, paralysis of the body caused by trauma to the spinal cord, cerebral contusion, amyotrophic lateral sclerosis, Huntington's disease, brain tumor, retinal degeneration, and the like can be treated with a regenerative medicine approach.

Alternatively, by supplying a suitable amount of neurodifferentiation-inducing agent (neurodifferentiation-inducing peptide) to cellular material that has been temporarily or permanently resected from the body, i.e., living tissue or cell clusters (for example, a culture product of somatic stem cells), a target peptide motif (BC-box-related sequence, etc.) can be transferred efficiently from outside the cells into the cytoplasm (more preferably, the nucleus) thereof, and neurons can be efficiently generated thereby. This means that large amounts of the desired neurons can be produced in the cellular material. Furthermore, by returning the neurons that were produced in large amounts or cellular material (living tissues and cell clusters) containing the produced neurons once again to the body (typically a diseased area requiring nerve regeneration), the same therapeutic efficacy can be obtained as when the neurodifferentiation-inducing agent (neurodifferentiation-inducing peptide) is administered directly to the body.

It is clear from the above explanation that, in a different aspect, the present invention can provide cells, cell clusters, and living tissues that are useful for treating neurological disorders and wherein differentiation to neurons has been induced by using any of the neurodifferentiation-inducing peptides of the abovementioned configurations disclosed herein.

Moreover, a polynucleotide coding for the neurodifferentiation-inducing peptide of the present invention can be used as a material for so-called gene therapy. For example, the neurodifferentiation-inducing peptide of the present invention can be expressed constantly in the body (cells) by incorporating a gene (typically a DNA segment or RNA segment) coding for the neurodifferentiation-inducing peptide into a suitable vector, and transfecting a target site therewith. Therefore, a polynucleotide (DNA segment, RNA segment, etc.) coding for the neurodifferentiation-inducing peptide of the present invention is useful as a drug for the prevention or treatment of a neurological disease in the abovementioned patients, etc.

At least one amino acid residue can be amidated in the construct for transferring a foreign substance (i.e., an artificially synthesized peptide) wherein the foreign substance is a peptide provided by the present invention such as the abovementioned neurodifferentiation-inducing peptide that is presented as a typical example. The structural stability (protease resistance) of the peptide in the cytoplasm and nucleus can be increased by amidation of the carboxyl group of an amino acid residue (typically the C-terminal amino acid residue of a peptide chain).

It is desirable for the total number of amino acid residues in the peptide chain constituting the artificial peptide to be 1000 or fewer (preferably, 600 or fewer, and particularly preferably 300 or fewer, e.g., 50 or fewer). Such a short peptide can be easily configured by chemical synthesis methods, and therefore can be easily supplied to a test sample containing the target eukaryotic cells.

It should also be noted that the conformation (three-dimensional structure) of the peptide is not particularly limited, but preferably it is a straight chain or helix from the standpoint of its not easily becoming an immunogen (antigen).

It should also be noted that as an artificial peptide preferably all of the amino acid residues are L-amino acids, but provided the desired function inherent in the carrier peptide fragment and peptide motif is not lost, part or all of the amino acid residues can be replaced by D-amino acids.

Moreover, an additional sequence that normally cannot occur in these sequences can be partly included therein provided the desired function inherent in the carrier peptide fragment and peptide motif is not lost. For example, an amino acid sequence can be configured with a structure wherein several amino acid residues functioning as a linker (for example, glycine residues) can be positioned between the carrier peptide fragment and the foreign peptide motif.

Among artificial peptides (constructs for transferring a foreign substance) to be used, those with a relatively short peptide chain can easily be produced by conventional chemical synthesis methods. For example, a either prior art publicly known solid phase or liquid phase synthesis method can be used. Solid phase synthesis using Boc (t-butyloxycarbonyl) or Fmoc (9-fluoroenylmethoxycarbonyl) as an amine protecting group is preferred. In other words, a peptide chain with the desired amino acid sequence and modifications (C-terminal amidation, etc.) can be synthesized by solid phase synthesis using a commercially available peptide synthesizer (e.g., one obtainable from PerSeptive Biosystems, Applied Biosystems, etc.).

Alternatively, the artificial peptide (construct for transferring a foreign substance) can be synthesized using genetic engineering methods. This approach is preferred for producing a polypeptide with a relatively long peptide chain. In other words, a DNA nucleotide sequence (including the ATG start codon) that codes for the amino acid sequence of the desired artificial peptide is synthesized. Then a recombinant vector suitable for a host cell is configured with a genetic construct for expression that comprises the DNA and various regulatory elements (including a promoter, ribosome binding site, terminator, enhancer, and a cis-element for controlling the level of expression) to express the amino acid sequence in the host cell.

Using conventional techniques this recombinant vector is transferred to designated host cells (for example, yeast cells, insect cells, plant cells, or animal (mammal) cells), and the host cells, or an individual or tissue containing the cells is cultured under designated conditions. The target polypeptide can be expressed and produced in the cells thereby. Furthermore, a peptide comprising the target amino acid sequence can be obtained by isolating and purifying the polypeptide from the host cells (or from the culture medium if it is secreted). Using conventional techniques this recombinant vector is transferred to a designated host cell (for example, yeast, insect cell, plant cell, or mammalian cell), and the host cell, or an individual or tissue containing the cells is cultured under prescribed conditions. The target polypeptide can be expressed and produced in the cells thereby. Then the target peptide (i.e., construct for transferring a foreign substance) can be obtained by isolating and purifying the polypeptide from the host cells (or from the culture medium if it is secreted).

It should be noted that the method for configuring the recombinant vector and the method for transferring the configured recombinant vector to a host cell, etc., can utilize methods conventionally used in the fields without modification, and because those methods themselves are not a characterizing feature of the present invention, the detailed explanation thereof is omitted herein.

For example, a fusion protein expression system can be used for efficient, large volume production in host cells. More specifically, first the gene (DNA) coding for the amino acid sequence of the target peptide is prepared by chemical synthesis, and the synthesized gene is inserted at a suitable site in a suitable fusion protein expression vector (for example, a GST (glutathione S-transferase) fusion protein expression vector such as the pET series provided by Novagen and the pGEX series provided by Amersham Biosciences). Then the host cells (typically E. coli) are transformed by the vector. The resulting transformant is cultured to prepare the target fusion protein. Next the protein is extracted and purified. Then the resulting purified is cleaved by a designated enzyme (protease) and the freed target peptide fragment (i.e., the designed artificial peptide) is recovered by a method such as affinity chromatography. The target construct for transferring a foreign substance (artificial peptide) can be produced using this kind of prior art and publicly known fusion protein expression system (for example, the GST/His system provided by Amersham Biosciences can be utilized).

Alternatively, template DNA for use in a cell-free protein synthesis system (i.e., a synthetic gene fragment containing a nucleotide sequence coding for the amino acid sequence of the target artificial peptide) can be prepared, and in vitro synthesis of the target polypeptide can be carried out by employing a so-called cell-free protein synthesis system using the various compounds necessary for peptide synthesis (ATP, RNA polymerase, amino acids, etc.). References concerning a cell-free protein synthesis system include the papers by Shimizu et al. (Shimizu et al., Nature Biotechnology, 19, 751-755 (2001)), and Madin et al. (Madin et al., Proc. Natl. Acad. Sci. USA, 97(2), 559-564 (2000)). When the present application was filed there were already many companies carrying out polypeptide production on consignment based on the technology disclosed in these documents, and cell-free protein synthesis kits were commercially available (for example the wheat germ cell-free protein synthesis kit PROTEIOS® obtainable from Toyobo Co., Ltd., in Japan).

Therefore, if an amino acid sequence (for example, the BC-box-related sequence noted above) corresponding to the peptide motif that is the object of transfer into the cytoplasm (preferably, the nucleus) can be determined, and a peptide chain can be designed that combines the same with the cell membrane-permeating carrier peptide fragment represented by abovementioned SEQ ID NO: 1, the intended artificial peptide can easily be synthesized and produced by a cell-free protein synthesis system based on its amino acid sequence. For example, the peptide can easily be produced with the PURESYSTEM® from Japan's Post Genome Institute Co., Ltd.

None of the carrier peptide fragments disclosed herein is particularly limited, and can be used in the method for transferring a foreign substance into a eukaryotic cell. However, the efficiency of transferring the foreign substance can differ for each individual fragment (amino acid sequence) depending on the type of eukaryotic cell.

For example, if the target cell for foreign substance transfer is a stem cell (including an iPS cell) from a human or nonhuman mammal, a carrier peptide fragment comprising the amino acid sequence represented by SEQ ID NO: 4 or SEQ ID NO: 6 (or a modified sequence thereof) has particularly high foreign substance transfer efficiency, and the use thereof is preferred.

Therefore, as a particularly preferred mode of the method for transferring a foreign substance disclosed herein, the present invention provides a method for transferring a foreign substance of interest from outside a human or nonhuman mammalian stem cell (particularly, an ES cell, iPS cell or somatic stem cell) into the cytoplasm of the cell (more preferably, also into the nucleus) by using a carrier peptide fragment comprising the amino acid sequence of SEQ ID NO: 4, SEQ ID NO: 6, or a modified amino acid sequence thereof formed by the substitution, deletion and/or addition (insertion) of 1, 2, or 3 amino acid residues in the amino acid sequence as the abovementioned carrier peptide fragment. The carrier peptide fragment comprising an amino acid sequence of the abovementioned two sequence identification numbers (4 and 6) is preferred for transferring a protein (typically about 300 to 1000 (for example, about 300 to 600) amino acid residues), a polypeptide of fewer than 300 amino acid residues, or a peptide motif with 100 or fewer (especially 50 or fewer) amino acid residues particularly into an iPS cell or ES cell.

Several examples concerning the present invention are described below, but the present invention is by no means limited to the items presented in these examples.

### Example 1

### Preparation of Construct for Transferring a Foreign Substance

A total of fourteen types of peptides (Sample No. 1 to Sample No. 14) described below were produced using a peptide synthesizer. Table 1 shows the amino acid sequences of these synthetic peptides.

TABLE 1

| | | | Total |
|---|---|---|---|
| | | | amino |
| Sample | | | acid |
| No. | Amino acid sequence | | residues |
| 1 | (FITC-Acp)-RSRKYTSWYVALKR | (SEQ ID | 14 |
| | | NO: 1) | |
| 2 | (FAM)-MAKSIRSKHRRQMRMMKRE | (SEQ ID | 19 |
| | | NO: 2) | |
| 3 | (FITC-Acp)-MARRRRHRGPRRPRPP | (SEQ ID | 16 |
| | | NO: 3) | |
| 4 | (FITC-Acp)-GRCRRLANFGPRKRRRRRR | (SEQ ID | 19 |
| | | NO: 4) | |
| 5 | (FITC-Acp)-RRRKRNRDARRRRRKQ | (SEQ ID | 16 |
| | | NO: 5) | |
| 6 | (FAM)-MQRKPTIRRKNLRLRRK | (SEQ ID | 17 |
| | | NO: 6) | |
| 7 | (FITC-Acp)-IMRRRGL | (SEQ ID | 7 |
| | | NO: 7) | |
| 8 | (FITC-Acp)-KKLKKRNK | (SEQ ID | 8 |
| | | NO: 8) | |
| 9 | (FAM)-RRRANNRRR | (SEQ ID | 9 |
| | | NO: 9) | |
| 10 | (FAM)-RKKRKKK | (SEQ ID | 7 |
| | | NO: 10) | |
| 11 | (FAM)-KRKGKLKNKGSKRKK | (SEQ ID | 15 |
| | | NO: 11) | |
| 12 | (FAM)-SKRLSSRARKRAAKRRLG | (SEQ ID | 18 |
| | | NO: 12) | |
| 13 | (FAM)-KRPRRRPSRPFRKP | (SEQ ID | 14 |
| | | NO: 13) | |
| 14 | (FITC-Acp)-RSRKYTSWYVALKRTLKERCLQVVRSLVK | (SEQ ID | 29 |
| | | NO: 94) | |

As shown in Table 1, Sample Nos. 1 to 13 are synthetic peptides comprising the carrier peptide fragments of SEQ ID NOS: 1 to 13. Moreover, Peptide No. 14 is configured to have the abovementioned VHL peptide motif (SEQ ID NO: 93: TLKERCLQVVRSLVK) as the foreign substance on the C-terminal end of the carrier peptide fragment represented by SEQ ID NO: 1).

It should also be noted that in each peptide the carboxyl group (—COOH) on the C-terminal amino acid is amidated (—CONH₂). Both peptides were synthesized using solid phase synthesis (Fmoc method) using a commercially available peptide synthesizer (Intavis AG) and following the instruction manual. It should also be noted that a detailed explanation of the mode of use of the peptide synthesizer itself has been omitted herein because it is not a characterizing feature of the present invention.

A construct for transferring a foreign substance was prepared by bonding a fluorescent dye as the foreign substance to the N-terminal ends of the abovementioned synthetic peptides that were obtained.

More specifically, as a fluorescent dye in Sample Nos. 2, 6, and 9 to 13 commonly used FAM (i.e., C₂₁H₁₂O₇: 5(6)-carboxyfluorescein, molecular weight 376.3) was bonded directly to the N-terminal end of the abovementioned peptides in a conventional method.

Alternatively, as a fluorescent dye in Sample Nos. 1, 3 to 5, 7, 8, and 14 commonly used FITC (i.e., C₂₁H₁₁NO₅S: fluorescein isothiocyanate, molecular weight 389.4) was bonded indirectly via the well-known linker Acp, in other words, 6-aminohexanoic acid (6-aminocaprioic acid, molecular weight 131.2) to the N-terminal end of above-mentioned peptide in a conventional method.

The sample peptides prepared in this way were each diluted in PBS (phosphate buffered saline) to prepare a total of fourteen types of sample solutions with a sample (peptide) concentration of 1 mM.

### Example 2

### Evaluation of Cell Membrane Permeability Function of Each Sample (Construct) (1)

Human neonate foreskin fibroblasts (ATCC Catalogue No. CRL-2097) were used as the eukaryotic cells, and the cell membrane permeability capability of the samples (constructs for transferring a foreign substance) obtained in Example 1 above was investigated.

More specifically, the abovementioned fibroblasts were cultured in a liquid mixture of 90% Eagle MEM culture medium (containing 0.1% nonessential amino acids, 2 mM L-glutamine, 1 mM sodium pyruvate, and 1.5 g/L sodium hydrogen carbonate) and 10% serum (FBS) as the culture medium.

The cultured cells were trypsinized for 1 min at 37° C. with a 0.25% trypsin solution. After the abovementioned treatment, the trypsin was deactivated with FBS-containing medium, and a cell suspension (test sample for foreign substance transfer) was prepared by adjusting the cell concentration to approximately 5×10⁴ cells/mL with the culture medium.

Next, 0.5 mL of the abovementioned cell suspension was placed in a designated cell culturing container (culture slide with a surface coating of 0.1% gelatin), and the cells were incubated overnight at 37° C. in a 5% CO₂ atmosphere. After incubation the medium was replaced with fresh culture medium, and the 1 mM sample solutions of each of the samples prepared in abovementioned Example 1 were added to a cell culture container so that the final sample concentration (peptide concentration) would be 1 μM. In this case 0.5 μL of sample solution was added to the container. Then the samples were incubated at 37° C. for either 1 or 4 hours in a 5% CO₂ atmosphere.

The cells after 1 hour of culture and the cells after 4 hours of culture were each rinsed in PBS and fixed with methanol (on ice for 10 min).

Next the methanol-fixed samples (cells) were mounted using Prolong® Gold Antifade Reagent (Invitrogen) that contains the nuclear stain DAPI (4′,6-diamidino-2-phenylindole). Then the localization within the cells of the peptide bonded to the fluorescent dye (i.e., fluorescently labeled peptide) in each test sample (i.e., the abovementioned mounted test samples after methanol fixation) was investigated using a confocal scanning laser microscope. Part of the results are presented herein as micrographs.

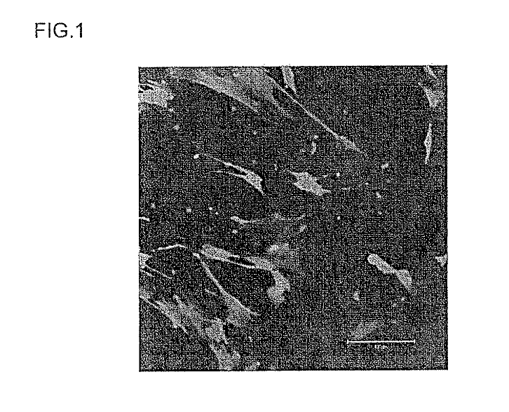

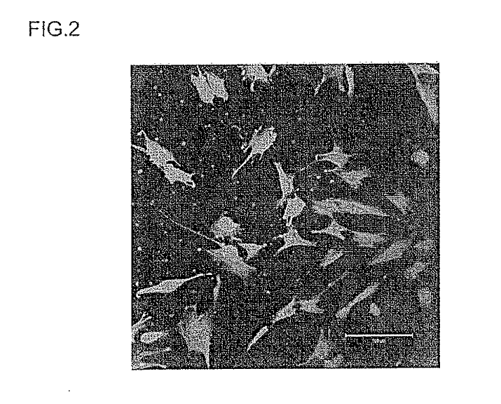

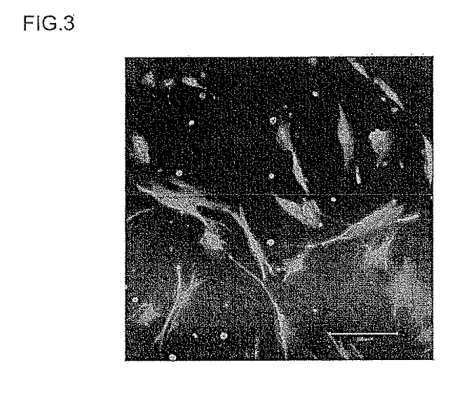

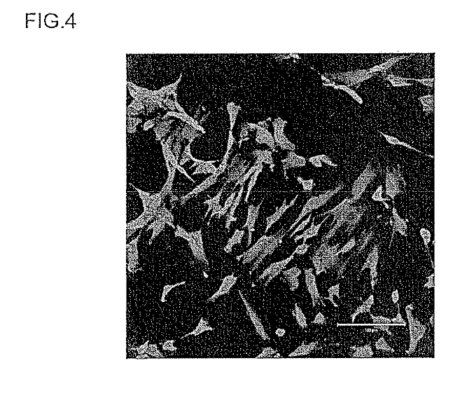

In other words, FIG. 1 is a micrograph showing the results after 1 hour of culture in a cell test sample to which abovementioned Sample No. 14 was added. FIG. 2 is a micrograph showing the results after 1 hour of culture in a cell test sample to which above-mentioned Sample No. 4 was added. FIG. 3 is a micrograph showing the results after 1 hour of culture in a cell test sample to which abovementioned Sample No. 5 was added. FIG. 4 is a micrograph showing the results after 1 hour of culture in a cell test sample to which abovementioned Sample No. 6 was added.

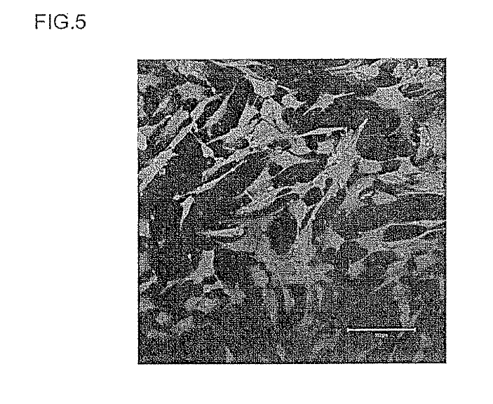

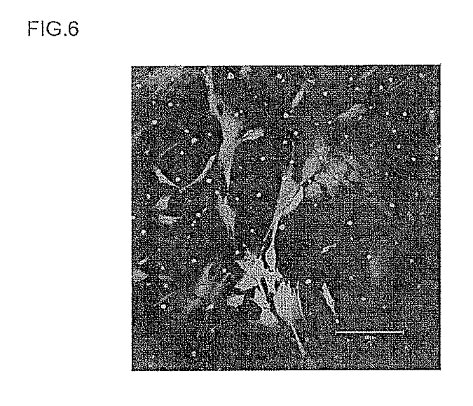

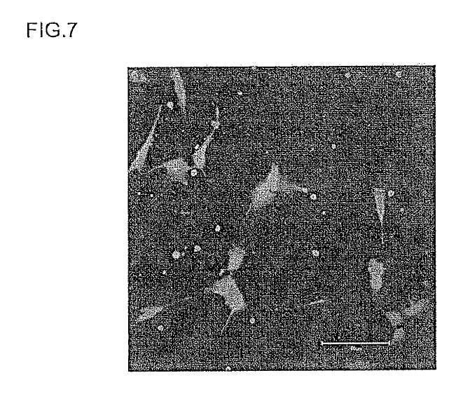

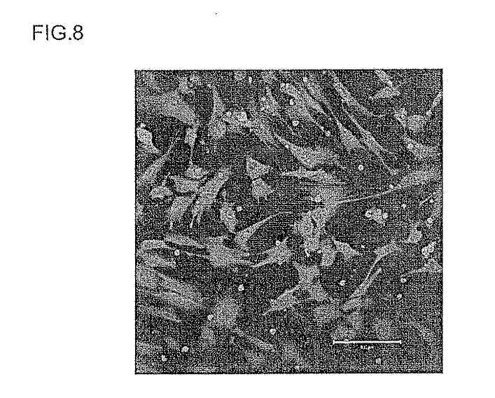

Moreover, FIG. 5 is a micrograph showing the results after 4 hours of culture in a cell test sample to which abovementioned Sample No. 14 was added. FIG. 6 is a micrograph showing the results after 4 hours of culture in a cell test sample to which above-mentioned Sample No. 2 was added. FIG. 7 is a micrograph showing the results after 4 hours of culture in a cell test sample to which abovementioned Sample No. 3 was added. FIG. 8 is a micrograph showing the results after 4 hours of culture in a cell test sample to which abovementioned Sample No. 4 was added.

As the micrographs clearly show, it was confirmed that Sample Nos. 2, 3, 4, 5, 6, and 14 penetrated the cell membrane from outside the cell rapidly after they were added, and were transferred into the cytoplasm. In particular, Sample Nos. 14 (identical to Sample No. 1), 4, and 6 which used the amino acid sequences of SEQ ID NOS: 1, 4 and 6, respectively, as the carrier peptide fragment were found to have extremely high foreign substance (in this case, a fluorescent dye and VHL peptide motif) transfer efficiency toward somatic cells such as fibroblasts.

In addition, from the results of the nuclear staining by DAPI it was confirmed that part of these samples that had been transferred into the cytoplasm had translocated into the nucleus (i.e., were transferred into the nucleus).

Although the micrographs are not presented here, nearly the same results as in Sample No. 14 were found in Sample Nos. 1 and 9 to 13. In contrast, with Sample Nos. 7 and 8 almost no passage through the cell membrane from outside and transfer of the foreign substance (i.e., sample peptide) into the cells was found.

### Example 3

### Evaluation of Cell Membrane Permeability Function of Sample No. 1 and Sample No. 2 (2)

The target cells for transferring the foreign substance were changed from human neonate foreskin fibroblasts to human iPS cells (human induced pluripotent stem cells), and the cell membrane permeability capability of the two samples (constructs for transferring a foreign substance) obtained in Example 1 above was investigated. It should also be noted that the iPS cells (cell line 201B2-082008KU) and the mouse embryonic fibroblast feeder cells (cell line SNL 76/7, hereinafter “MEF”) used in this example were provided by the Yamanaka Research Laboratory of the Institute for Frontier Medical Sciences, Kyoto University (professor Shinya Yamanaka).

First the obtained MEF were inactivated by a mitomycin C treatment (3 hours) and then trypsinised in a 0.25% trypsin solution containing 1 mM EDTA. After the above treatment, the trypsin was deactivated with culture medium containing FBS, and the MEF were adjusted to a suitable cell density using D-MEM medium (Dulbecco's Modified Eagle Medium: Gibco) containing MEF culture medium (7% FBS: Gibco), 2 mM L-glutamine (Gibco), 50 units/mL penicillin, and 50 μg/mL streptomycin (Gibco), and the abovementioned MEF were seeded onto a culture container (in the form of a culture slide or plate) with a surface coating of 0.1% gelatin. In this case, the MEF were seeded so that the cell density would be approximately 1.25×10⁵ cells/mL. Next, the abovementioned cell culture containers were incubated overnight at 37° C. in a 5% CO₂ atmosphere.

Thereafter, the feeder cells were prepared by removing the MEF culture medium and rinsing with PBS.

Separately, CTK solution (0.25% trypsin solution containing 0.1 mg/mL collagenase IV (Gibco), 1 mM calcium chloride, and 20% KSR (KnockOut® Serum Replacement)) was added to the obtained iPS cell line, the MEF were peeled therefrom, and the cells were rinsed with PBS.

Next, 1 mL hESC culture medium (i.e., human ES cell medium, in this case, DMEM/F12 culture medium (Gibco) containing 20% KSR (Gibco), 2 mM L-glutamine (Gibco), 0.1% nonessential amino acids (Gibco), 0.1 mM 2-mercaptoethanol (Gibco), 50 units/mL penicillin, and 50 μg/mL streptomycin (Invitrogen, 4 ng/mL bFGF (basic fibroblast growth factor)) was added, the iPS cells were peeled off using a cell scraper, and the colony was broken apart by gentle pipetting.

A suspension of iPS cells obtained in this manner was seeded onto the feeder cells in the culture containers that had been prepared as described above. Then, the above-mentioned hESC medium was added, and the cell culture containers were incubated overnight at 37° C. in a 5% CO₂ atmosphere.

After incubation overnight, the culture medium was removed from the culture containers, and the abovementioned hESC medium to which one of the 1 mM sample solutions prepared in abovementioned. Example 1 had been added to make a final sample concentration (pipetted concentration) of 1 μM was added to the culture containers. Then the samples were incubated at 37° C. for either 1 or 4 hours in a 5% CO₂ atmosphere.

The cells after 1 hour of culture and cells after 4 hours of culture were each rinsed in PBS (phosphate buffered saline) and fixed with methanol (on ice for 10 min).

Next, the same treatment as in Example 2 was performed, and the localization within the iPS cells of the peptide that was bonded to the fluorescent dye (i.e., fluorescently labeled) was investigated using a confocal scanning laser microscope. Part of the results are presented herein as micrographs.

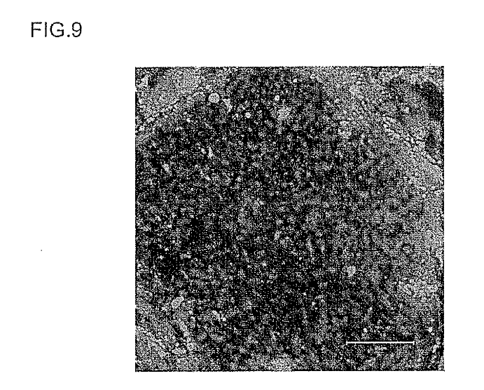

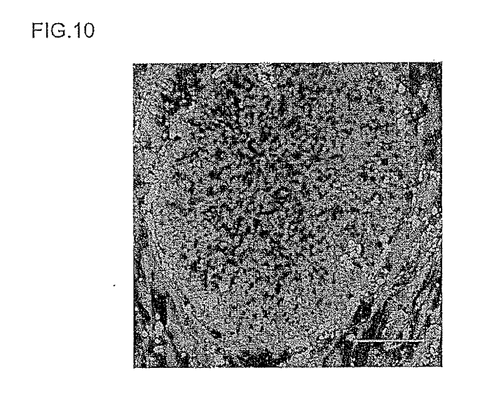

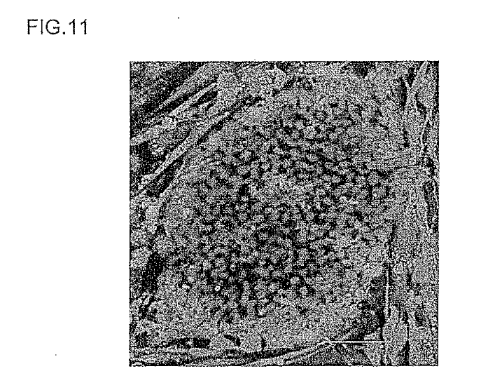

In other words, FIG. 9 is a micrograph showing the results after 1 hour of culture in a cell test sample to which abovementioned Sample No. 14 was added. FIG. 10 is a micrograph showing the results after 1 hour of culture in a cell test sample to which abovementioned Sample No. 4 was added. FIG. 11 is a micrograph showing the results after 1 hour of culture in a cell test sample to which abovementioned Sample No. 6 was added.

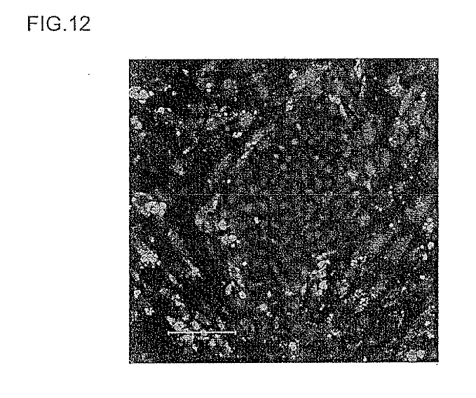

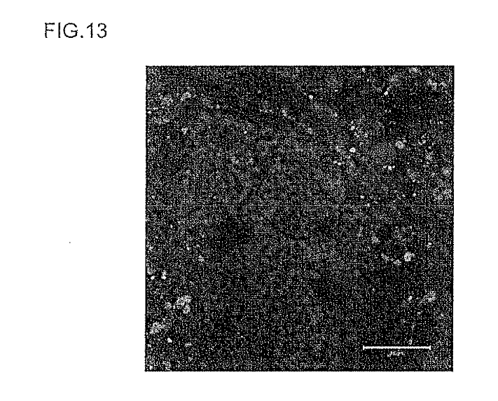

FIG. 12 is a micrograph showing the results after 1 hour of culture in a cell test sample to which abovementioned Sample No. 7 was added. FIG. 13 is a micrograph showing the results after 1 hour of culture in a cell test sample to which abovementioned Sample No. 8 was added.

As the micrographs clearly show, it was confirmed that Sample Nos. 4, 6, and 14 penetrated the cell membrane from outside the cell rapidly after they were added and were transferred into the cytoplasm of iPS cells. In particular, it was confirmed that Sample Nos. 4 and 6, which used the amino acid sequences of SEQ ID NOS: 4 and 6, respectively, as the carrier peptide fragment have an extremely high foreign substance transfer efficiency toward stem cells such as iPS cells. In addition, from the results of the nuclear staining by DAN it was confirmed that part of these samples that had been transferred into the cytoplasm had translocated into the nucleus (i.e., were transferred into the nucleus).

In contrast, with Sample Nos. 7 and 8 almost no passage through the cell membrane from outside and transfer of the foreign substance (i.e., sample peptide) into iPS cells was found.

Although the micrographs are not presented here, nearly the same results as in Sample No. 14 were found in the other samples.

Specific examples of the present invention have been described in detail above, but these are merely exemplary and by no means limit the scope of the claims herein. The technology disclosed in the claims includes various changes to and variations of the specific examples presented above.

The present invention enables the transfer of a foreign substance of interest having a designated function into a human or other mammalian stem cell (for example, a somatic stem cell and induced pluripotent stem cell) or other target cell. Thereby it is possible to transform the target cell in accordance with the foreign substance (peptide, etc.) to be transferred, and for example, bring about the differentiation thereof to a specific cell type (nerve cell, bone cell, muscle cell, skin cell, etc.).

The present invention provides an artificially prepared construct for transferring a foreign substance of interest from outside a eukaryotic cell (in particular, various animal cells typified by human and nonhuman mammalian cells that do not have a cell wall) into the cytoplasm (preferably, the nucleus as well) thereof. By utilizing this construct a foreign substance of interest can be effectively transferred into a target cell, and cells wherein the foreign substance has been transferred into the cytoplasm (preferably the nucleus), as well as organs and other body tissues comprising cells that contain the foreign substance can be obtained thereby.

1. A method for transferring a foreign substance of interest from outside an induced pluripotent stem cell (iPS cell) at least into the cytoplasm of the iPS cell, comprising the steps of:
preparing a construct comprising:
a carrier peptide fragment consisting of the amino acid sequence set forth in SEQ ID NO: 6, and
a foreign substance of interest that is bonded directly or indirectly via a linker to an N-terminus or C-terminus of the carrier peptide fragment;
preparing feeder cells and seeding the feeder cells onto a culture container containing culture medium;
incubating the feeder cells at least overnight;
preparing iPS cells and seeding the iPS cells onto the feeder cells after the incubation in the culture container;
adding the construct to the iPS cells on the feeder cells in the culture container; and
incubating the iPS cells on the feeder cells at least one hour after adding the construct so as to introduce the construct into the iPS cell from outside of the cell by cell membrane permeability of the construct itself.

2. The method according to claim 1, wherein the foreign substance is any organic compound selected from the group consisting of peptides, nucleic acids, dyes, and drugs.

3. The method according to claim 2, wherein the foreign substance is a peptide, and the construct is a synthetic peptide comprising the carrier peptide fragment and a peptide fragment serving as the foreign substance that is bonded directly or indirectly via a linker to the N-terminus or C-terminus of the carrier peptide fragment.

4. The method according to claim 1, wherein the iPS cells are human induced pluripotent stem cells.

5. The method according to claim 4, wherein the feeder cells are mouse embryonic fibroblast feeder cells.

6. The method according to claim 1, wherein the iPS cells on the feeder cells after adding the construct are incubated for at least 1 hour at 37° C.


SEQUENCE LISTING (94 sequences)

SEQ ID NO: 1 - protein, 14 aa, Artificial sequence
  Synthetic peptide
RSRKYTSWYVALKR

SEQ ID NO: 2 - protein, 19 aa, Artificial sequence
  Synthetic peptide
MAKSIRSKHRRQMRMMKRE

SEQ ID NO: 3 - protein, 16 aa, Artificial sequence
  Synthetic peptide
MARRRRHRGPRRPRPP

SEQ ID NO: 4 - protein, 19 aa, Artificial sequence
  Synthetic peptide
GRCRRLANFGPRKRRRRRR

SEQ ID NO: 5 - protein, 16 aa, Artificial sequence
  Synthetic peptide
RRRKRNRDARRRRRKQ

SEQ ID NO: 6 - protein, 17 aa, Artificial sequence
  Synthetic peptide
MQRKPTIRRKNLRLRRK

SEQ ID NO: 7 - protein, 7 aa, Artificial sequence
  Synthetic peptide
IMRRRGL

SEQ ID NO: 8 - protein, 8 aa, Artificial sequence
  Synthetic peptide
KKLKKRNK

SEQ ID NO: 9 - protein, 9 aa, Artificial sequence
  Synthetic peptide
RRRANNRRR

SEQ ID NO: 10 - protein, 7 aa, Artificial sequence
  Synthetic peptide
RKKRKKK

SEQ ID NO: 11 - protein, 15 aa, Artificial sequence
  Synthetic peptide
KRKGKLKNKGSKRKK

SEQ ID NO: 12 - protein, 18 aa, Artificial sequence
  Synthetic peptide
SKRLSSRARKRAAKRRLG

SEQ ID NO: 13 - protein, 14 aa, Artificial sequence
  Synthetic peptide
KRPRRRPSRPFRKP

SEQ ID NO: 14 - protein, 15 aa, Artificial sequence
  Synthetic peptide
PLQELCRQRIVAAVG

SEQ ID NO: 15 - protein, 15 aa, Artificial sequence
  Synthetic peptide
TLQHFCRLAINKCTG

SEQ ID NO: 16 - protein, 15 aa, Artificial sequence
  Synthetic peptide
TLQHLCRKTVNGHLD

SEQ ID NO: 17 - protein, 15 aa, Artificial sequence
  Synthetic peptide
SLQHICRTVICNCTT

SEQ ID NO: 18 - protein, 15 aa, Artificial sequence
  Synthetic peptide
SLQYICRAVICRCTT

SEQ ID NO: 19 - protein, 15 aa, Artificial sequence
  Synthetic peptide
SLQYLCRFVIRQYTR

SEQ ID NO: 20 - protein, 15 aa, Artificial sequence
  Synthetic peptide
SLQHLCRFRIRQLVR

SEQ ID NO: 21 - protein, 15 aa, Artificial sequence
  Synthetic peptide
SLQDLCCRAVVSCTP

SEQ ID NO: 22 - protein, 15 aa, Artificial sequence
  Synthetic peptide
SLQDLCCRTIVSCTP

SEQ ID NO: 23 - protein, 15 aa, Artificial sequence
  Synthetic peptide
SLQHICRMSIRRVMS

SEQ ID NO: 24 - protein, 15 aa, Artificial sequence
  Synthetic peptide
SLKHLCRKALRSFLT

SEQ ID NO: 25 - protein, 15 aa, Artificial sequence
  Synthetic peptide
TLLSLCRVAVRRALG

SEQ ID NO: 26 - protein, 15 aa, Artificial sequence
  Synthetic peptide
PLAHLCRLRVRKAIG

SEQ ID NO: 27 - protein, 15 aa, Artificial sequence
  Synthetic peptide
SLTHLCRLEIRSSIK

SEQ ID NO: 28 - protein, 15 aa, Artificial sequence
  Synthetic peptide
TLLESSARTILHNRI

SEQ ID NO: 29 - protein, 15 aa, Artificial sequence
  Synthetic peptide
NLQDLCRIKIRQCIG

SEQ ID NO: 30 - protein, 15 aa, Artificial sequence
  Synthetic peptide
SLQHLCRCALRSHLE

SEQ ID NO: 31 - protein, 15 aa, Artificial sequence
  Synthetic peptide
SLKHLCRLKIRKCMG

SEQ ID NO: 32 - protein, 15 aa, Artificial sequence
  Synthetic peptide
PLQELCRQRIVATVG

SEQ ID NO: 33 - protein, 15 aa, Artificial sequence
  Synthetic peptide
PLAHLCRLRVRKAIG

SEQ ID NO: 34 - protein, 15 aa, Artificial sequence
  Synthetic peptide
SLQHLCRMSIRRVMP

SEQ ID NO: 35 - protein, 15 aa, Artificial sequence
  Synthetic peptide
SLQDLCCRAVVSCTP

SEQ ID NO: 36 - protein, 15 aa, Artificial sequence
  Synthetic peptide
SLQYLALTALITPKK

SEQ ID NO: 37 - protein, 15 aa, Artificial sequence
  Synthetic peptide
SLQFLALTVYTDFLR

SEQ ID NO: 38 - protein, 15 aa, Artificial sequence
  Synthetic peptide
SLQYLALRVYTNGLR

SEQ ID NO: 39 - protein, 15 aa, Artificial sequence
  Synthetic peptide
SLQLLALVAYTNGIR

SEQ ID NO: 40 - protein, 15 aa, Artificial sequence
  Synthetic peptide
SLQYLALLAHQNGLR

SEQ ID NO: 41 - protein, 15 aa, Artificial sequence
  Synthetic peptide
SLQYLALQVYLKDGG

SEQ ID NO: 42 - protein, 15 aa, Artificial sequence
  Synthetic peptide
SLQYLAIKAWARQQL

SEQ ID NO: 43 - protein, 15 aa, Artificial sequence
  Synthetic peptide
SLQYLALKVVSDVRS

SEQ ID NO: 44 - protein, 15 aa, Artificial sequence
  Synthetic peptide
SLQYLALTVVSHVRS

SEQ ID NO: 45 - protein, 15 aa, Artificial sequence
  Synthetic peptide
SLQFLALVVVQQNGR

SEQ ID NO: 46 - protein, 15 aa, Artificial sequence
  Synthetic peptide
SLQFLALRVVQEGKN

SEQ ID NO: 47 - protein, 15 aa, Artificial sequence
  Synthetic peptide
SLQFLALQVVQKGHG

SEQ ID NO: 48 - protein, 15 aa, Artificial sequence
  Synthetic peptide
SLQFLCLRVLHGQQE

SEQ ID NO: 49 - protein, 15 aa, Artificial sequence
  Synthetic peptide
SLQFLCLRQLQHVQN

SEQ ID NO: 50 - protein, 15 aa, Artificial sequence
  Synthetic peptide
SLQFLCLRQLQHVQS

SEQ ID NO: 51 - protein, 15 aa, Artificial sequence
  Synthetic peptide
SLQYLCLRQLQHVQT

SEQ ID NO: 52 - protein, 15 aa, Artificial sequence
  Synthetic peptide
SLQFICLRQLQHVQA

SEQ ID NO: 53 - protein, 15 aa, Artificial sequence
  Synthetic peptide
SLQFLCLRVIYGPEE

SEQ ID NO: 54 - protein, 15 aa, Artificial sequence
  Synthetic peptide
TLQFLCLQAYLRGRK

SEQ ID NO: 55 - protein, 15 aa, Artificial sequence
  Synthetic peptide
TLQLLCLRAYIKFCR

SEQ ID NO: 56 - protein, 15 aa, Artificial sequence
  Synthetic peptide
SLQCIAGGQVLASWF

SEQ ID NO: 57 - protein, 15 aa, Artificial sequence
  Synthetic peptide
SLQCMSAGMLLGRWF

SEQ ID NO: 58 - protein, 15 aa, Artificial sequence
  Synthetic peptide
SLQCMAGGAVLAVWF

SEQ ID NO: 59 - protein, 15 aa, Artificial sequence
  Synthetic peptide
SLQCIAGGQVLASWF

SEQ ID NO: 60 - protein, 15 aa, Artificial sequence
  Synthetic peptide
SLQCRAGGTLLAVWF

SEQ ID NO: 61 - protein, 15 aa, Artificial sequence
  Synthetic peptide
SLRCMAGGAVLALWF

SEQ ID NO: 62 - protein, 15 aa, Artificial sequence
  Synthetic peptide
SLQCKAGGVVLANWF

SEQ ID NO: 63 - protein, 15 aa, Artificial sequence
  Synthetic peptide
SLRCMAGGAVLALWF

SEQ ID NO: 64 - protein, 15 aa, Artificial sequence
  Synthetic peptide
SLRCMAGGAVLALWF

SEQ ID NO: 65 - protein, 15 aa, Artificial sequence
  Synthetic peptide
SLRCMAGGAVLALWF

SEQ ID NO: 66 - protein, 15 aa, Artificial sequence
  Synthetic peptide
SLQCIAGGAVLAIWF

SEQ ID NO: 67 - protein, 15 aa, Artificial sequence
  Synthetic peptide
SLQCLSATQVLKEFL

SEQ ID NO: 68 - protein, 15 aa, Artificial sequence
  Synthetic peptide
SLQCRAMRRILLHVI

SEQ ID NO: 69 - protein, 15 aa, Artificial sequence
  Synthetic peptide
SLQCLAAKQVLLKCF

SEQ ID NO: 70 - protein, 15 aa, Artificial sequence
  Synthetic peptide
SLQCLAAKSVLLSCF

SEQ ID NO: 71 - protein, 17 aa, Artificial sequence
  Synthetic peptide
SLQYLALKALVTPKKIK

SEQ ID NO: 72 - protein, 17 aa, Artificial sequence
  Synthetic peptide
SLQYLALAALITPKKIK

SEQ ID NO: 73 - protein, 17 aa, Artificial sequence
  Synthetic peptide
SLQYLALTALIKPKKIK

SEQ ID NO: 74 - protein, 17 aa, Artificial sequence
  Synthetic peptide
SLQYLALTALITPKKIK

SEQ ID NO: 75 - protein, 17 aa, Artificial sequence
  Synthetic peptide
SLQYLALKALVTPTRTR

SEQ ID NO: 76 - protein, 17 aa, Artificial sequence
  Synthetic peptide
SLQYLALTALVAPKKTK

SEQ ID NO: 77 - protein, 17 aa, Artificial sequence
  Synthetic peptide
TLQLLALRAVVKARSRK

SEQ ID NO: 78 - protein, 17 aa, Artificial sequence
  Synthetic peptide
TLQFLALKAVVKVKRNK

SEQ ID NO: 79 - protein, 17 aa, Artificial sequence
  Synthetic peptide
TLQYLALTAWVGAKKRK

SEQ ID NO: 80 - protein, 17 aa, Artificial sequence
  Synthetic peptide
SLQFLALKALISERRHR

SEQ ID NO: 81 - protein, 17 aa, Artificial sequence
  Synthetic peptide
SLQFLALKALVGQSKRR

SEQ ID NO: 82 - protein, 17 aa, Artificial sequence
  Synthetic peptide
SLQYLALRAWVRVGKKK

SEQ ID NO: 83 - protein, 15 aa, Artificial sequence
  Synthetic peptide
PLMDLCRRSVRLALG

SEQ ID NO: 84 - protein, 15 aa, Artificial sequence
  Synthetic peptide
SLLHLSRLCVRHNLG

SEQ ID NO: 85 - protein, 15 aa, Artificial sequence
  Synthetic peptide
PLMDLCRRSIRSALG

SEQ ID NO: 86 - protein, 15 aa, Artificial sequence
  Synthetic peptide
SLQDLCCRAIVSCTP

SEQ ID NO: 87 - protein, 15 aa, Artificial sequence
  Synthetic peptide
ALFELCGRAVSAHMG

SEQ ID NO: 88 - protein, 11 aa, Artificial sequence
  Synthetic peptide
SLQCIAGGQVL

SEQ ID NO: 89 - protein, 11 aa, Artificial sequence
  Synthetic peptide
SLQHLCRLVIN

SEQ ID NO: 90 - protein, 11 aa, Artificial sequence
  Synthetic peptide
SLNKMCSNLLE

SEQ ID NO: 91 - protein, 14 aa, Artificial sequence
  Synthetic peptide
LFELCGRAVSAHMG

SEQ ID NO: 92 - protein, 15 aa, Artificial sequence
  Synthetic peptide
SLQHLCRLVINRLVA

SEQ ID NO: 93 - protein, 15 aa, Artificial sequence
  Synthetic peptide
TLKERCLQVVRSLVK

SEQ ID NO: 94 - protein, 29 aa, Artificial sequence
  Synthetic peptide
RSRKYTSWYVALKRTLKERCLQVVRSLVK